(19) United States
(12) Patent Application Publication
(10) Pub. No.: US 2011/0293660 A1
(43) Pub. Date: Dec. 1, 2011

(54) NOVEL METHOD

(76) Inventors: Bruno Rene Andre, Rixensart (BE); Benoit Paul Suzanne Champluvier, Rixensart (BE)

(21) Appl. No.: 13/147,827

(22) PCT Filed: Feb. 4, 2010
(86) PCT No.: PCT/EP2010/051340

§ 371 Date: Aug. 4, 2011

Related U.S. Application Data
(60) Provisional application No. 61/150,421, filed on Feb. 6, 2009.

(51) Int. Cl. A61K 39/145; C07K 1/14; C12N 9/24; A61P 37/04; C07K 1/36; C12N 7/04; A61P 31/16; C12N 7/02; C12N 9/12

(52) U.S. Cl. 424/209.1; 435/239; 530/427; 435/200; 530/358; 435/194; 435/236

(57) ABSTRACT

The present invention relates to a method for purifying a virus, or a viral antigen thereof, comprising at least the following steps: a) obtaining a fluid comprising the virus, or a viral antigen thereof, and b) purifying the fluid by at least one density gradient ultracentrifugation step, wherein the ratio of the amount of virus, or viral antigen thereof, present in the fluid over the density gradient volume is less than 1, less than 0.8, less than 0.6 and less than 0.4.

### STATEMENT REGARDING FEDERALLY SPONSORED RESEARCH

Aspects of this invention were made with United States government support pursuant to Contract # HHSO 100200600011C, from the Department of Health and Human Services; the United States government may have certain rights in the invention.

### TECHNICAL FIELD

The present invention relates to a method for purifying viruses, or viral antigens thereof, and more particularly for purification of viruses, or viral antigens, produced by cell culture. The invention provides a purification method for improving the virus yield.

### TECHNICAL BACKGROUND

Due to the vast number of diseases caused by viruses, virology has been an intensively studied field. There has always been the demand to produce viruses efficiently in order to isolate and purify viral proteins, to generate vaccines, to prepare analytical tools, or to provide viruses for laboratory studies.

Recently, cell culture-based technologies as an alternative to the traditional egg-based production systems have been developed.

Cell culture systems appear as a suitable alternative mode of vaccine preparation in particular, simpler, flexible, consistent, allowing to improve possibilities of up-scaling vaccine production capacities and thus to reach large quantities of virus, if needed, in particular, in case of a pandemic threat or a terrorist attack.

However, after production, the cell culture-produced virus requires to be recovered from the cell culture, and, when appropriate, to be purified. Methods for purifying viruses are known in the art. U.S. Pat. No. 6,008,036, for instance, discloses the use of chromatographic matrices for purifying cell culture-based viruses. Some other methods are also described. One particular preferred method, for example, employs sucrose gradient centrifugation (U.S. Pat. No. 6,048,537). Such processes, while they allow to increase the virus, or viral antigen, purity present the major drawback of providing a low virus yield, as virus material is lost along the way. More recently, methods relying on improved and optimized density gradient ultracentrifugation have been provided. For example, US 2008/0274138 discloses a method for virus purification using centrifugation on a sugar gradient established by the addition of two or more buffered sugar layers of different concentration. WO 2008/073490 provides a method for purifying viruses by ultracentrifugation on a linear reverse glycerol-potassium tartrate gradient. Both of these methods rely on the elaboration of a particular gradient which is much more complex than density gradients previously known in the art.

Therefore, a need remains for providing alternative and improved methods for virus recovery and purification which are simple to implement.

### SUMMARY OF THE INVENTION

The method according to the present invention provides a simple solution to limit the virus loss when implementing a density gradient ultracentrifugation step during the purification process, and, therefore, increase the virus yield.

In a first aspect of the present invention, there is provided a method for purifying a virus, or a viral antigen thereof, comprising at least the following steps:
a) obtaining a fluid comprising the virus or a viral antigen thereof, and
b) purifying the fluid by at least one density gradient ultracentrifugation step, wherein the ratio of the amount of virus, or viral antigen thereof, present in the fluid, over the density gradient volume is less than 1, less than 0.8, less than 0.6 and less than 0.4

In a second aspect of the present invention, there is provided a method for purifying a virus, or a viral antigen thereof, produced by cell culture, comprising at least one step of density gradient ultracentrifugation step, wherein the ratio of the amount of virus, or viral antigen thereof, present in a fluid comprising the virus, or a viral antigen thereof, and to be loaded on the density gradient, over the density gradient volume is less than 1, less than 0.8, less than 0.6 and less than 0.4.

In a third aspect, there is provided a method for the preparation of a vaccine comprising at least the step of admixing the virus obtained according to the present invention with a pharmaceutically acceptable carrier.

### DESCRIPTION OF DRAWINGS

FIG. 1: Influence of the ratio HA amount/rotor volume (mg/ml) on HA yield (%) after a sucrose gradient ultracentrifugation step. HA yield represents the percentage of HA recovered after the sucrose gradient ultracentrifugation step (performed with the indicated rotor), which was calculated by measuring the HA amount before and after the ultracentrifugation by SRD assay. The HA yield values were plotted against the ratio HA amount/rotor volume values.

### DETAILED DESCRIPTION

The present invention relates to a method for purifying viruses, in particular, viruses produced by cell culture, that can be applied to both small and large scale virus production. The method involves, in particular, an improved step of density gradient ultracentrifugation allowing to reach a higher virus yield. The virus preparation resulting from the method according to the present invention may be further purified by using standard techniques employed for virus purification. The virus prepared according to the present invention can be used for any purpose, including, for instance, purification of viral proteins, analytical assays, infection of host cells, diagnostic purposes or therapeutic or prophylactic uses such as vaccination and clinical administration.

According to the invention, the ratio of the amount of virus, or viral antigen thereof, present in the fluid to be loaded on the gradient over the volume of said gradient is an important factor which needs to be taken into account. Indeed, the inventors unexpectedly observed that varying the volume of the gradient density, while keeping the amount of virus constant, results in a better virus yield. In particular, doubling the density gradient volume results in a 2-fold increase of the virus yield.

Accordingly, in one embodiment of the invention, the ratio of the amount of virus, or viral antigen thereof, present in the fluid, expressed in weight, such as mg, over the density gradient volume expressed in ml, is less than 1, in particular, less than 0.8, more particularly less than 0.6 and even less than 0.4 mg of virus/ml of gradient. Virus weight may also be expressed in higher units, such as g, or lower units, such as μg. Gradient volume may also be expressed as L or μl. However, ratio calculation according to the invention, must rely on using the values for weight and volume indicated in equivalent units, i.e. g/L, mg/ml, or μg/μl. It is to be understood by “equivalence”, in the context of the present invention, the same order of magnitude.

For example, if a rotor has a volume capacity of y ml, then the volume of gradient which can be loaded on said rotor is y ml. If a virus fluid comprises x mg of viruses, then the ratio x mg of virus/y ml of gradient, according to the invention is, suitably, lower than 1, more suitably, lower than 0.8, even more suitably less than 0.6 and even less than 0.4 mg of virus/ml of gradient.

In a specific embodiment, the virus yield after a density gradient ultracentrifugation, i.e. the percentage of antigen which is recovered after the ultracentrifugation, in particular, the yield of HA antigen from influenza virus obtained when using a ratio according to the method of the invention, for instance, a ratio ranging from 0.4 to 1, is higher than 50%, in particular, higher than 60%, suitably, higher than 70%, more suitably, higher than 80%, and even higher than 85%, as measured by SRD (Single Radial Immunodiffusion).

In another embodiment, the virus yield obtained when using a ratio according to the method of the invention, ranging from 0.4 to 1, is at least 1.5, in particular 2, suitably 2.5 and more suitably 3 times higher, as compared to that obtained with a ratio greater than 1, for instance a ratio ranging from 1.3 to 2.

In one embodiment, the ratio according to the method of the invention is adjusted by increasing the volume of the density gradient.

The amount of virus can be analyzed by any known in the art techniques. For instance, the presence of a virus can be quantified by monitoring and measuring the detection of one of the virus constituents, suitably, a specific viral antigen. As an illustrative technique may be cited the analysis of a specific viral antigen by Western-blot analysis. When considering the influenza virus, the antigen HA can be detected and quantified by Western-blot analysis with an anti-HA antibody. A distinct assay for measuring the content of HA is the SRD (Single Radial Immunodiffusion) assay, which is a technique familiar to a person skilled in the art (J. M. Wood et al.: An improved single radial immunodiffusion technique for the assay of influenza haemagglutinin antigen: adaptation for potency determination of inactivated whole virus and subunit vaccines. J. Biol. Stand. 5 (1977) 237-247; J. M. Wood et al., International collaborative study of single radial diffusion and immunoelectrophoresis techniques for the assay of haemagglutinin antigen of influenza virus. J. Biol. Stand. 9 (1981) 317-330)).

Density gradient ultracentrifugation is a technique commonly used for purifying viruses. It is especially used in the vaccine manufacturing field. When a fluid comprising a virus is loaded on a density gradient and subjected to ultracentrifugation, viruses will migrate to a location in the gradient where their density is equivalent to the density of gradient, while contaminants will not be stopped in the gradient and will sediment. Sediments will be discarded and only gradient fractions containing the virus will be collected. Depending on the type of rotor used, a dynamic collection (during centrifugation) or a static collection (at rest) after reorientation of the gradient will be performed. According to the invention, the density gradient ultracentrifugation may be zonal or isopycnic. It may be performed in a continuous mode, in a semi-continuous mode or in successive batches.

The density gradient ultracentrifugation used according to the method of the present invention is not limited in any way and the invention encompasses any type of ultracentrifugation, in any conditions, including any of the above conditions. For instance, no particular speed requirement is imposed by the method of the present invention. The appropriate centrifugation speed can be set according to the virus to be purified, the type of centrifuge, the type of rotors, as well as the features of the rotor. It can be any speed, provided that said speed allows the virus to enter the gradient and to reach its density within the gradient. By way of example, and as an indication only, when willing to purify influenza virus on a sucrose gradient, ultracentrifugation may be performed at 35000 rpm.

The present invention does not rely on the use of a specific density gradient, and can, thus, be applicable to any density gradient. The choice of the density gradient constituent is dependent on the virus which is to be purified and on the application which is intended for the resulting purified virus. For instance, enveloped viruses are less dense than non-enveloped viruses. This is known in the art. Specifically, depending on what purpose is the virus produced according to the method prepared for, a constituent which does not affect virus integrity or its biological activity will be used. For example, when the virus prepared according to the method of the invention is intended for vaccination, the constituent is chosen so as to maintain the immunogenicity of the virus, or of the viral antigen thereof. Sugar solutions, in particular sucrose, may be used to generate density gradients for use in the process according to the invention. Sucrose is particularly used to purify enveloped viruses, such as, but not limited to, influenza viruses. It is also a sugar frequently used in the field of vaccine manufacturing.

In a specific embodiment, the sugar used to create a density gradient according to the method of the present invention is sucrose. This is advantageous for the purification of products for human use when safety considerations need to be kept in mind. However, the invention also contemplates the use of other sugars, including alcohol sugars, such as, for instance, sorbitol, or hydrogenated sugars, linear sugars, and modified sugars or any other sugar provided that the sugar has a solubility in water sufficient to produce solutions with densities specified according to the type of virus to be purified. Alternatively, the gradient may also be prepared with potassium tartrate, which presents the advantage of reaching a gradient with a higher density, compared to sucrose gradient. Accordingly, potassium tartrate gradients are, in particular, suitably employed for purifying non-enveloped viruses.

Density gradients according to the present invention are not limited to sugar gradients. The present invention also contemplates, as other illustrative examples, the use of gradients of salts, such as, for instance, caesium chloride, which is suitable for the purification of both enveloped viruses and non-enveloped viruses.

The present invention is not limited to a particular concentration of the density gradients. The concentration range of the density gradient should be determined depending on the virus to be purified, in particular, depending on the virus density.

When the density gradient is a sucrose gradient, a typical density range for purifying viruses is 0-55% (w/v). The presence of a specific viral antigen at a certain range within the gradient can be monitored by standard techniques of protein detection, such as a Western-blot analysis using an antibody specific for the viral antigen. In the particular case of the influenza virus, the content of one of its surface antigen, the HA antigen, can be monitored by the SRD assay.

According to the invention, density gradients can be linear or discontinuous. They may also be pre-formed, i.e. formed before starting the centrifugation, or they may form during the centrifugation.

In a specific embodiment, the density gradient according to the method of the present invention is a linear gradient, in particular, a linear sucrose gradient 0-55% (v/w), suitably formed during the centrifugation, said centrifugation being performed, for instance, as a continuous-flow operation.

Although it is not required, density gradients may be, suitably, prepared in a buffered solution comprising salt at a physiological concentration, in particular, in a citrate-containing Phosphate Buffer Saline (PBS) solution, as this type of solution advantageously prevents virus aggregation.

The method of virus purification according to the invention is amenable to a wide range of viruses, including, but not limited to, adenoviruses, hepadnaviruses, herpes viruses, orthomyxoviruses, papovaviruses, paramyxoviruses, picornaviruses, poxviruses, reoviruses and retroviruses. In particular, the method of invention is suitable for enveloped viruses, such as myxoviruses. In one embodiment, the viruses produced by the method of the invention belong to the family of orthomyxoviruses, in particular, influenza virus.

Viruses or viral antigens may be derived from an orthomyxovirus, such as influenza virus. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase (PB1, PB2 and PA). Particularly suitable antigens include HA and NA, the two surface glycoproteins which determine the antigenic specificity of the Influenza subtypes.

Influenza virus is selected from the group consisting of human influenza virus, avian influenza virus, equine influenza virus, porcine (e.g. swine) influenza virus, feline influenza virus. Influenza virus is more particularly selected from strains A, B and C, preferably from strains A and B.

Influenza virus or antigens thereof may be derived from interpandemic (annual or seasonal) influenza strains. Alternatively, influenza virus or antigens thereof may be derived from strains with the potential to cause a pandemic outbreak (i.e., influenza strains with new hemagglutinin compared to hemagglutinin in currently circulating strains, or influenza strains which are pathogenic in avian subjects and have the potential to be transmitted horizontally in the human population, or influenza strains which are pathogenic to humans). Depending on the particular season and on the nature of the antigen included in the vaccine, the influenza virus or antigens thereof may be derived from one or more of the following hemagglutinin subtypes: H1, H2, H3, H4, H5, H6, H7, H8, H9, H10, H11, H12, H13, H14, H15 or H16. Preferably, the influenza virus or antigens thereof are from H1, H2, H3, H5, H7 or H9 subtypes.

In the context of the present invention, a “fluid comprising a virus, or a viral antigen thereof”, is to be understood as any preparation which comprises a virus, or a viral antigen thereof, irrespective of the production system used. Non-limiting examples are fluids originating from cell culture, embryonated egg, such as allantoic fluid, baculovirus expression medium, or biological fluid. Biological fluids may be urine, blood, semen, spinal fluid, pulmonary fluid, bronchial lavage fluid or saliva from any species.

The cells which are used in the method according to the invention can in principle be any desired cell type of cells which can be cultured in cell culture and which can support virus replication. They can be both adherently growing cells or cells growing in suspension. They can be either primary cells or continuous cell lines. Genetically stable cell lines are preferred.

Mammalian cells are particularly suitable, for example, human, hamster, cattle, monkey or dog cells.

A number of mammalian cell lines are known in the art and include PER.C6, HEK cells, human embryonic kidney cells (293 cells), HeLa cells, CHO cells, Vero cells, and MDCK cells.

Suitable monkey cells are, for example, African green monkey cells, such as kidney cells as in Vero cell line. Suitable dog cells are, for example, kidney cells as in MDCK cell line.

Suitable mammalian cell lines for growing influenza virus include MDCK cells, Vero cells, or PER.C6 cells. These cell lines are all widely available, for instance, from the American Type Cell Culture (ATCC) collection.

According to a specific embodiment, the method of the invention uses MDCK cells. The original MDCK cell line is available from the ATCC as CCL-34, but derivatives of this cell line may also be used, such as the MDCK cells adapted to growth in suspension (WO 1997/37000).

Alternatively, cell lines for use in the invention may be derived from avian sources, such as chicken, duck, goose, quail or pheasant. Avian cell lines may be derived from a variety of developmental stages including embryonic, chick and adult. In particular, cell lines may be derived from the embryonic cells, such as embryonic fibroblasts, germ cells, or individual organs, including neuronal, brain, retina, kidney, liver, heart, muscle, or extraembryonic tissues and membranes protecting the embryo. Chicken embryo fibroblasts (CEF) may be used. Examples of avian cell lines include avian embryonic stem cells (WO01/85938) and duck retina cells (WO05/042728). In particular, the EB66® cell line derived from duck embryonic stem cells is contemplated in the present invention (WO 2008/129058). Other suitable avian embryonic stem cells include the EBx cell line derived from chicken embryonic stem cells, EB45, EB14 and EB14-074 (WO2006/108846). This EBx cell line presents the advantage of being a genetically stable cell line whose establishment has been produced naturally and did not require any genetic, chemical or viral modification. These avian cells are particularly suitable for growing influenza viruses.

According to a particular embodiment, the method of the invention uses EB66 cells.

Cell culture conditions (temperature, cell density, pH value, etc . . . ) are variable over a very wide range owing to the suitability of the cells employed and can be adapted to the requirements of particular virus growth conditions details. It is within the skilled person's capabilities to determine the appropriate culture conditions, as cell culture is extensively documented in the art (see, for example, Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

In a specific embodiment, host cells used in the method described in the present invention are cultured in serum-free and/or protein-free media. A “serum-free medium” (SFM) means a cell culture medium ready to use that does not require serum addition allowing cell survival and cell growth. This medium may not necessarily be chemically defined and may contain hydrolyzates of various origin, from plant for instance. Such serum-free media present the advantage that contamination with viruses, mycoplasma or unknown infectious agents can be ruled out. “Protein-free” is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such culture naturally contain protein themselves.

Serum-free media are commercially available from numerous sources, for instance, VP SFM (Invitrogen Ref 11681-020), Opti-Pro (Invitrogen, Ref 12309-019), or EX-CELL (JHR Bioscience).

Cells may be grown in various ways, for instance, in suspension, or adhering to surfaces, including growth on microcarriers. Culturing can be done in dishes, flasks, roller bottles, or in bioreactors, or combinations thereof, using batch, fed-batch, semi-continuous or continuous systems, such as perfusion systems. Typically, cells are scaled-up from a master or working cell bank vial through various sizes of flasks or roller bottles and finally to bioreactors. In one embodiment, the cells employed according to the method of the invention are cultured on microcarrier beads in a serum-free medium in a stirred-bioreactor and the culture medium is provided by perfusion.

In an alternative embodiment, cells are cultured in suspension in a batch mode.

Prior to infection with the virus, cells are cultured around 37° C., more suitably at 36.5° C., at a pH ranging from 6.7 to 7.8, suitably around 6.8 to 7.5, and more suitably around 7.2

According to the method of the invention, the production of cell culture-based viruses includes generally the steps of inoculating the cultured cells with the viral strain to be produced and cultivating the infected cells for a desired period of time so as to allow virus replication.

In order to produce large quantities of cell-produced viruses, it is preferred to inoculate cells with the desired virus strain once cells have reached a high density. Usually, the inoculation is performed when the cell density is at least around 1.5×10⁶ cells/ml, suitably, around 3×10⁶ cells/ml, more suitably, around 5×10⁶ cells/ml, even more suitably 7×10⁶ cells/ml, or even higher. The optimal cell density for obtaining the highest virus production may vary according to the cell type used for the virus propagation.

The inoculation is carried out at an MOI (Multiplicity Of Infection) of about 10⁻¹ to 10⁻⁷, suitably about 10⁻² to 10⁻⁶, and more suitably, about 10⁻⁵.

The temperature and pH conditions for virus infection may vary. Temperature may range from 32° C. to 39° C. depending on the virus type. For Influenza virus production, cell culture infection may vary depending on the strain which is produced. Influenza virus infection is suitably performed at a temperature ranging from 32° C. to 35° C., suitably at 33° C. In one embodiment, the virus infection occurs at 33° C. In an alternative embodiment, the virus infection takes place at 35° C. Proteases, typically trypsin, may be added to the cell culture depending on the virus strain, to allow viral replication. The protease can be added at any suitable stage during the culture.

Once infected, cells may release into the culture medium newly formed virus particles, due to spontaneous lysis of host cells, also called passive lysis. Therefore, in one embodiment, cell-based viral harvest may be provided any time after virus inoculation by collecting the cell culture medium or supernatant. In a particular embodiment, the cell culture medium is collected by perfusion. This mode of harvesting is particularly suitable when it is desired to harvest cell-based virus at different time points after virus inoculation, and pooling the different harvests, if needed.

Alternatively, after virus infection, cell-based virus may be harvested by employing external factor to lyse host cells, also called active lysis. However, contrary to the previous one, such a harvesting mode requires that the cell-based viral harvest be collected at a single time point, as actively lysing the cells will immediately terminate the cell culture.

Methods that can be used for active cell lysis are known. Useful methods in this respect are for example, freeze-thaw, solid shear, hypertonic and/or hypotonic lysis, liquid shear, high pressure extrusion, detergent lysis, or any combination thereof.

According to one embodiment, cell-based viral harvest may be provided any time after virus inoculation by collecting the cell culture medium or supernatant, lysing the inoculated cells or both.

In the sense of the present invention, when referring to a cell culture which has been inoculated and infected with a virus, the expression “cell culture medium”, or “culture medium”, or “medium” and the term “supernatant” are to be considered synonyms. “Medium” and “supernatant”, in the context of an infected cell culture, are to be understood as fluids comprising a virus, or a viral antigen thereof.

Before collection of culture medium or supernatant, cell infection may last for 2 to 10 days. The optimal time to collect the cell-based fluid comprising a virus, or a viral antigen thereof, is based on the determination of the infection peak. For example, the CPE (CytoPathic Effect) can be measured by monitoring the morphological changes occurring in host cells after virus inoculation, including cell rounding, disorientation, swelling or shrinking, death, detachment from the surface. The detection of a specific viral antigen can be monitored by standard techniques of protein detection, such as a Western-blot analysis and proceed to the collection when the desired detection level is achieved. In the particular case of influenza virus, the content of HA can be monitored any time post-inoculation of the cells with the virus, by the SRD assay, as described above.

After collection, viruses may be purified. For instance, purification may include a number of different filtration, concentration and/or other separation steps such as ultrafiltration, ultracentrifugation (including gradient ultracentrifugation), chromatography (such as ion exchange chromatography) and adsorption steps in a variety of combinations. A clarification step may be required in order to separate the virus from cellular material contaminant, in particular, floating cells or cell debris.

Therefore, the fluid comprising a virus, or a viral antigen thereof, according to the invention is not limited to crude fluids, but also contemplates fluids which comprise partially purified viruses. The term “crude” in the sense of the present invention means that no purification has been performed on the fluid comprising the virus, or a viral antigen thereof, after its collection, and, thus, may contain any kind of contaminants to varying extents. As a non-limiting example, when the virus has been produced by inoculating cells with said virus and when, after infection, newly formed viruses are released to the cell culture medium or supernatant, then said culture medium, which comprises the virus, designates an example of a crude fluid. Another example of a crude fluid which may be cited is the allantoic fluid harvested after inoculation of virus onto embryonated eggs and virus cultivation. Accordingly, the terms “partially purified” encompass any intermediate purification status, i.e. a fluid which has been the subject of any purification step, for instance, any of the steps mentioned above, individually, or in any combination.

In one embodiment, the fluid according to the invention is the culture medium collected after infection of the cells with the virus of interest.

In another suitable embodiment, the fluid according to the invention has been clarified. This clarification may be done by filtration. Alternatively, centrifugation and/or filtration may be combined together, in any order, for achieving the desired clarification level of the virus preparation. Suitable filters may utilize cellulose filters, regenerated cellulose filters, cellulose fibers combined with inorganic filter aids, cellulose filter combined with inorganic filter aids and organic resins, or any combination thereof, and polymeric filters. Although not required, a multiple filtration process may be carried out, like a two- or three-stage process consisting, for instance, in sequentially and progressively removing impurities according to their size, using filters with appropriate nominal pore size, in particular, filters with decreasing nominal pore size, allowing to start removing large precipitates and cell debris. In addition, single stage operations employing a relatively tight filter or centrifugation may also be used for clarification. More generally, any clarification approach including, but not limited to, dead-end filtration, depth filtration, microfiltration, or centrifugation, which provide a filtrate of suitable clarity to not foul the membrane and/or resins in subsequent steps, will be acceptable to use in the clarification step of the present invention. In one specific embodiment, the viral clarification step is performed by depth filtration, in particular, using a three-stage train filtration composed, for example, of three different depth filters with nominal porosities of 5 μm-0.5 μm-0.2 μm. In another embodiment, the viral harvest is pre-clarified by centrifugation and then clarified by depth filtration, for instance, using a filtration train composed of two different filters with nominal porosities of 0.5 μm-0.2 μm.

Although it is not required, it may be suitable to concentrate the fluid comprising the virus, or a viral antigen thereof, prior to loading it on the density gradient according to the invention, in order to reduce the fluid volume to be loaded. Accordingly, the present invention also contemplates a fluid comprising a virus, or a viral antigen thereof, which has been concentrated, prior to loading on the density gradient. Therefore, the fluid comprising a virus, or a viral antigen thereof, may be subjected to ultrafiltration (sometimes referred to as diafiltration when used for buffer exchange), for instance on an 750 kD membrane, for concentrating the virus and/or buffer exchange. This step is particularly advantageous when the virus to be purified is diluted, as is the case when pooling viral harvest collected by perfusion over a few days post-inoculation. The process used to concentrate the virus according to the method of the present invention can include any filtration process where the concentration of virus is increased by forcing diluent to be passed through a filter in such a manner that the diluent is removed from the virus suspension whereas the virus is unable to pass through the filter and thereby remains in concentrated form in the virus preparation.

Ultrafiltration may comprise diafiltration which is an ideal way for removal and exchange of salts, sugars, non-aqueous solvents, removal of material of low molecular weight, of rapid change of ionic and/or pH environments. Microsolutes are removed most efficiently by adding solvent to the solution being ultrafiltered at a rate equal to the ultrafiltration rate. This washes microspecies from the solution at a constant volume, isolating the retained virus. Diafiltration is particularly advantageous when a downstream step requires that a specific buffer be used in order to get an optimal reaction. Concentration and diafiltration may be implemented at any suitable step of the purification process, when it is wanted to remove undesirable compounds, such as sucrose, after a sucrose gradient ultracentrifugation, or such as formaldehyde, after a step of virus inactivation with formaldehyde. The system is composed of three distinct process streams: the feed solution (comprising the virus), the permeate and the retentate. Depending on the application, filters with different pore sizes may be used. The filter composition may be, but is not limited to, regenerated cellulose, polyethersulfone, polysulfone, or derivatives thereof. The membranes can be flat sheets (also called fait screens) or hollow fibers.

In one embodiment, the fluid comprising a virus, or a viral antigen thereof, has been concentrated by ultrafiltration/diafiltration, in particular, prior to loading it on the density gradient from the ultracentrifugation step performed according to the method of the present invention.

The purification method according to the present invention may include additional steps, in addition to the optimized step of density gradient ultracentrifugation as herein claimed, which is based on a specified ratio of the amount of virus, or viral antigen thereof, to be loaded, over the density gradient volume. These steps may be implemented before or after proceeding to said optimized density gradient ultracentrifugation step. Specifically, the virus preparation obtained after using the density gradient ultracentrifugation step according to the present invention may be further purified, by implementing any one of the previously cited virus purification techniques, such as filtration, ultracentrifugation (including gradient ultracentrifugation), chromatography (such as ion exchange chromatography) and adsorption steps in a variety of combinations.

In one embodiment, the method of the present invention further comprises at least one step selected from the group of: filtration, ultrafiltration/diafiltration, ultracentrifugation and chromatography, or any combination thereof. Depending on the purity level that is desired, the above steps may be combined in any way.

In one embodiment, the method according to the present invention further comprises a second step of ultracentrifugation, possibly a density gradient ultracentrifugation, in particular, a sucrose gradient ultracentrifugation. This additional ultracentrifugation step may occur before or after the optimized density gradient ultracentrifugation of the present invention.

In a specific embodiment, the virus purified according to the density gradient ultracentrifugation of the present invention is the subject of a second ultracentrifugation step, said step being possibly a sucrose gradient ultracentrifugation step.

Alternatively, it is also possible to further purify viruses by chromatography, including ion exchange, anionic or cationic, chromatography, size exclusion, such as gel filtration or gel permeation, chromatography, hydrophobic interaction chromatography, hydroxyapatite or any combination thereof. As mentioned above, the chromatography steps may be implemented in combination with other purifications steps, such as density gradient ultracentrifugation.

At the end of the virus purification, the virus preparation may be suitably subjected to sterile filtration, as is common in processes for pharmaceutical grade materials, such as immunogenic compositions or vaccines, and known to the person skilled in the art. Such sterile filtration can for instance suitably be performed by filtering the preparation through a 0.22 μm filter. After sterile preparation, the virus or viral antigens are ready for clinical use, if desired.

The present invention further relates to viruses and to compositions comprising viruses or viral antigens obtainable by a method according to the invention and to their use in medicine. They can be formulated by any known method to give a vaccine for administration to humans or animals. Therefore, immunogenic compositions, such as vaccines, comprising viruses or viral antigens of this type are also contemplated by the present invention.

The immunogenic compositions, in particular vaccines, may generally be formulated in a sub-virion form, e.g. in the form of a split virus, where the lipid envelope has been dissolved or disrupted, or in the form of one or more purified viral proteins (subunit vaccine). As an alternative, the immunogenic compositions may include a whole virus, e.g. a live attenuated whole virus, or an inactivated whole virus.

Accordingly, a fluid comprising a virus, or a viral antigen thereof, in the sense of the present invention, may comprise whole viruses, whether live attenuated or inactivated, split viruses and purified viral antigens. The fluid according to the invention may also comprise viral antigens expressed recombinantly.

The present invention also contemplates that a splitting step be implemented, at any time, during the purification method of the invention, whether it is before, during or after the step (b) of said method.

Methods of splitting viruses, such as influenza viruses, are well known in the art (WO02/28422). Splitting of the virus is carried out by disrupting or fragmenting whole virus whether infectious (wild-type or attenuated) or non-infectious (inactivated) with a disrupting concentration of a splitting agent. Splitting agents generally include agents capable of breaking up and dissolving lipid membranes. Traditionally, split influenza virus was produced using a solvent/detergent treatment, such as tri-n-butyl phosphate, or diethylether in combination with Tween™ (known as “Tween-ether” splitting) and this process is still used in some production facilities. Other splitting agents now employed include detergents or proteolytic enzymes or bile salts, for example sodium deoxycholate. Detergents that can be used as splitting agents include cationic detergents e.g. cetyl thrimethyl ammonium bromide (CTAB), other ionic detergents, e.g. sodium lauryl sulphate (SLS), taurodeoxycholate, or non-ionic detergents such as Tween or Triton X-100, or combination of any two or more detergents.

In one embodiment, the splitting agent is deoxycholate. In another embodiment, the splitting agent is Triton X-100. In a further embodiment, the method according to the invention uses a combination of Triton X-100 and sodium lauryl sulfate as splitting agents

The splitting process may be carried out as a batch, continuous or semi-continuous process. When implemented in batch, the split virus may require an additional step of purification, such as a chromatography step.

In one embodiment, the fluid comprising a virus, or a viral antigen thereof, is purified by a density gradient ultracentrifugation according to the invention and the purified virus, optionally, further purified by a second density gradient ultracentrifugation, and the purified virus, or viral antigen thereof, is split in a batch mode, in particular with Triton X-100.

It is not necessary to implement a splitting step as such, as it is possible to perform the splitting simultaneously to another purification step. In particular, a splitting agent may be added to a density gradient. Such an embodiment is particularly suitable, when it is desired to minimize the total number of steps of the method of the invention, as it allows, within a single operation, to both purify and split the virus. Hence, in one embodiment, the density gradient of the optimized density gradient ultracentrifugation step used according to the method of the present invention for purifying a fluid comprising a virus, or a viral antigen thereof, in particular, a sucrose gradient, additionally comprises a splitting agent.

Alternatively, the splitting agent may be added to the density gradient of the additional ultracentrifugation step when a further density gradient ultracentrifugation is implemented in addition to the optimized density gradient ultracentrifugation of the present invention.

For the safety of vaccines, it may be necessary to reduce infectivity of the virus suspension along different steps of the purification process. The infectivity of a virus is determined by its capacity to replicate on a cell line. Therefore, the method according to the present invention, optionally, includes at least one virus inactivation step, occurring at any time. The inactivation may be performed by using BPL (Beta-Propiolactone) at any suitable step of the method. In one embodiment, the method according to the invention further comprises at least one BPL treatment step. In another embodiment, the method according to the invention further comprises at least one BPL treatment step and at least one formaldehyde treatment step. Formaldehyde and BPL may be used sequentially, in any order, for instance, formaldehyde is used after the BPL.

Immunogenic compositions of the present invention, including vaccines, can optionally contain the additives customary for vaccines, in particular substances which increase the immune response elicited in a patient who receives the composition, i.e. so-called adjuvants.

In one embodiment, immunogenic compositions are contemplated, which comprise a virus or viral antigen of the present invention admixed with a suitable pharmaceutical carrier. In a specific embodiment, they comprise an adjuvant.

Adjuvant compositions may comprise an oil-in-water emulsion which comprise a metabolisable oil and an emulsifying agent. In order for any oil-in-water composition to be suitable for human administration, the oil phase of the emulsion system comprises a metabolisable oil. The meaning of the term metabolisable oil is well known in the art. Metabolisable can be defined as ‘being capable of being transformed by metabolism’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others.

A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil by virtue of the fact that it is an intermediate in the biosynthesis of cholesterol (Merck index, 10th Edition, entry no.8619). In a further embodiment of the invention, the metabolisable oil is present in the immunogenic composition in an amount of 0.5% to 10% (v/v) of the total volume of the composition.

The oil-in-water emulsion further comprises an emulsifying agent. The emulsifying agent may suitably be polyoxyethylene sorbitan monooleate. Further, said emulsifying agent is suitably present in the vaccine or immunogenic composition 0.125 to 4% (v/v) of the total volume of the composition.

The oil-in-water emulsion of the present invention optionally comprise a tocol. Tocols are well known in the art and are described in EP0382271. Suitably may be a tocol is alpha-tocopherol or a derivative thereof such as alpha-tocopherol succinate (also known as vitamin E succinate). Said tocol is suitably present in the adjuvant composition in an amount 0.25% to 10% (v/v) of the total volume of the immunogenic composition.

The method of producing oil-in-water emulsions is well known to the person skilled in the art. Commonly, the method comprises mixing the oil phase (optionally comprising a tocol) with a surfactant such as a PBS/TWEEN80™ (or polysorbate 80) solution, followed by homogenisation using a homogenizer. A suitable method comprises passing the mixture twice through a syringe needle would be suitable for homogenising small volumes of liquid. Alternatively, the emulsification process in microfluidiser (M110S Microfluidics machine, maximum of 50 passes, for a period of 2 minutes at maximum pressure input of 6 bar (output pressure of about 850 bar)) could be adapted by the man skilled in the art to produce smaller or larger volumes of emulsion. The adaptation could be achieved by routine experimentation comprising the measurement of the resultant emulsion until a preparation was achieved with oil droplets of the required diameter.

In an oil-in-water emulsion, the oil and emulsifier should be in an aqueous carrier. The aqueous carrier may be, for example, phosphate buffered saline.

In particular, the oil-in-water emulsion systems of the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or suitably the Malvern Zetasizer 3000HS. A detailed procedure is given in Example 11.2. A first possibility is to determine the z average diameter ZAD by dynamic light scattering (PCS-Photon correlation spectroscopy); this method additionally give the polydispersity index (PDI), and both the ZAD and PDI are calculated with the cumulants algorithm. These values do not require the knowledge of the particle refractive index. A second mean is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

The adjuvant compositions may further comprise a Toll like receptor (TLR) 4 agonist. By “TLR4 agonist” it is meant a component which is capable of causing a signalling response through a TLR4 signalling pathway, either as a direct ligand or indirectly through generation of endogenous or exogenous ligand (Sabroe et al, JI 2003 p 1630-5). The TLR 4 may be a lipid A derivative, particularly monophosphoryl lipid A or more particularly 3 Deacylated monophoshoryl lipid A (3 D-MPL).

3D-MPL can be produced according to the methods disclosed in GB 2 220 211 A. Chemically it is a mixture of 3-deacylated monophosphoryl lipid A with 3, 4, 5 or 6 acylated chains. In particular, in the adjuvant compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)
OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)
OM 197 MP-Ac DP (3S—,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants. In addition, further suitable TLR-4 agonists are disclosed in US2003/0153532 and US2205/0164988.

The invention is particularly suitable for preparing influenza virus immunogenic compositions, including vaccines. Various forms of influenza virus are currently available. They are generally based either on live virus or inactivated virus. Inactivated vaccines may be based on whole virions, spilt virions or purified surface antigens (including HA). Influenza antigens can also be presented in the form of virosomes (nucleic acid-free viral-like liposomal particles).

Virus inactivation methods and splitting methods have been described above and are applicable to influenza virus.

Influenza virus strains for use in vaccines change from season to season. In the current inter-pandemic period, vaccines typically include two influenza A strains and one influenza B strain. Trivalent vaccines are typical, but higher valence, such as quadrivalence, is also contemplated in the present invention. The invention may also use HA from pandemic strains (i.e. strains to which the vaccine recipient and the general human population are immunologically naïve), and influenza vaccines for pandemic strains may be monovalent or may be based on a normal trivalent vaccine supplemented by a pandemic strain.

Compositions of the invention may include antigen(s) from one or more influenza virus strains, including influenza A virus and/or influenza B virus. In particular, a trivalent vaccine including antigens from two influenza A virus strains and one influenza B virus strain is contemplated by the present invention. Alternatively a quadrivalent vaccine including antigens from two influenza A virus strains and two influenza B virus strains is also within the scope of the present invention.

The compositions of the invention are not restricted to monovalent compositions, i.e. including only one strain type, i.e. only seasonal strains or only pandemic strains. The invention also encompasses compositions comprising a combination of seasonal strains and of pandemic strains. In particular, a quadrivalent composition, which may be adjuvanted, comprising three seasonal strains and one pandemic strain falls within the scope of the invention. Other compositions falling within the scope of the invention are a trivalent composition comprising two A strains and one B strain, such as H1N1, H3N2 and B strains, and a quadrivalent composition comprising two A strains and two B strains of a different lineage, such as H1N1, H3N2, B/Victoria and B/Yamagata.

HA is the main immunogen in current inactivated influenza vaccines, and vaccine doses are standardized by reference to HA levels, typically measured by SRD. Existing vaccines typically contain about 15 μg of HA per strain, although lower doses can be used, e.g. for children, or in pandemic situations, or when using an adjuvant. Fractional doses such as a half (i.e. 7.5 μg HA per strain) or a quarter have been used, as have higher doses, in particular, 3× or 9× doses. Thus immunogenic compositions of the present invention may include between 0.1 and 150 μg of HA per influenza strain, particularly, between 0.1 and 50 μg, e.g. 0.1-20 μg, 0.1-15 μg, 0.1-10 μg, 0.1-7.5 μg, 0.5-5 μg, etc. Particular doses include about 15, about 10, about 7.5, about 5 μg per strain, about 3.8 μg per strain and about 1.9 μg per strain.

Once an influenza virus has been purified for a particular strain, it may be combined with viruses from other strains to make a trivalent vaccine, for example, as described above. It is more suitable to treat each strain separately and to mix monovalent bulks to give a final multivalent mixture, rather than to mix viruses and degrade DNA and purify it from a multivalent mixture.

The invention will be further described by reference to the following, non-limiting, examples.

### Example 1

### Production of Influenza Virus in MDCK Cells and Purification by Sucrose Gradient Ultracentrifugation—Ha Yield Improvement by Increasing the Volume of the Sucrose Gradient (Experiments JP104, JP115, JP125, EFC3APA001, EFC3APA002 and DFC3APA003)

### 1. Virus Multiplication

The MDCK adherent cells were grown on microcarriers in perfusion culture mode in a 20 liter stirred-bioreactor scale at 36.5° C. After the growth phase, once the appropriate cell density was reached, above 5×10⁶ cells/ml (JP104, JP115, JP125 and EFC3APA001) or around 2.5×10⁶ cells/ml (EFC3APA002 and DFC3APA003), cells were inoculated with Influenza virus (Multiplicity of Infection of 1×10⁻⁵), Jiangsu B strain (JP104, JP115, JP125, EFC3APA001 and EFC3APA002) or Malaysia B strain (DFC3APA003) in perfusion mode and the temperature was switched to 33° C. (Jiangsu B strain) or to 35° C. (Malaysia B strain). Benzonase was added to the bioreactor at a final concentration of 1.5 Units/ml at day 3 and day 4 post-inoculation (JP125, EFC3APA001, EFC3APA002 and DFC3APA003). No benzonase was added in the experiments JP115 and JP104. The virus was harvested by perfusion at days 3 and 4 post-inoculation (JP115) or at days 3, 4 and 5 post-inoculation (JP125, JP104, EFC3APA001, EFC3APA002 and DFC3APA003). The perfusion harvests were pooled and stored at a temperature ranging from 2 to 8° C. until further processing.

### 2. Virus Purification

a) Viral harvests from all experiments were clarified on a filtration train composed of three different depth filters with nominal porosities of 5 μm-0.5 μm-0.2 μm. The clarified harvests were stored at a temperature ranging from 2 to 8° C. overnight.

b) The clarified harvests were then concentrated by ultrafiltration with a 750 kD hollow fiber membrane so as to get a final volume of around 2 liters, diafiltrated against 5 volumes of PBS containing 125 mM citrate and 0.001% Triton X-100-pH 7.4 and against 4 volumes of 10 mM Tris, 2 mM MgCl₂ pH 8, 100 μM CaCl₂, 0.001% Triton X-100-pH 8.

c) The retentates were removed from the ultrafiltration system and warmed up to 37° C. in a water bath. DNA degradation was performed by adding Benzonase™ (Merck) to the retentates at a final concentration of 270 Units/ml (JP115), or 135 Units/ml (JP104), or 200 Units/ml (JP125, EFC3APA001, EFC3APA002 and DFC3APA003) and the mixture was incubated 1 hour at 37° C.

d) Next, the ultrafiltration retentate was subjected to a sucrose gradient ultracentrifugation. In order to evaluate the impact of increasing the volume of the gradient, in some experiments (JP104, JP115 and JP125), the gradient volume was 400 ml, while in the other ones (EFC3APA001, EFC3APA002 and DFC3APA003), the gradient volume was 1.6 L, using, respectively, rotors with a volume capacity of 400 ml and rotors with a volume capacity of 1.6 L. The centrifuge was a Pilot System PKII continuous flow ultracentrifuge from Alfa Wasserman used with PK3 rotors. Sucrose gradient solutions prepared in PBS pH 7.4 containing 125 mM of citrate were loaded to the rotor and then accelerated to the rotational speed of 35000 rpm, so as to form a linear sucrose gradient (0-55% v/w). After continuous loading with the concentrated harvest (around 2 L for each experiment) at a pump flow rate of 1 L/h for 400 ml rotors and 4.1 L/h for 1.6 L rotors, the rotor was flushed with a PBS pH 7.4 containing 125 mM of citrate washing buffer, in a continuous way, to remove residual material which has not entered the gradient. After flushing, the rotor was decelerated and the ultracentrifuge was stopped, and fractions collection was performed.

Once all the volume to be purified was loaded onto the gradient, a banding time of 60 minutes allowed most of the virus to reach its density within the gradient. The viral particles were concentrated within a few fractions. The product fractions were in PBS pH 7.4 containing 125 mM citrate and sucrose. The purified whole virion was pooled from the percentage of sucrose ranging from approximately 28% to 52%. This range has been determined on the basis of profiles from SDS-PAGE and from Western Blot analysis using anti-HA and anti-MDCK antibodies. The whole virion pooled fractions were stored at a temperature ranging from 2 to 8° C., then diluted 9-10 fold in PO₄ 66 mM pH 7.4 (JP115 and JP104) or in PBS pH 7.4 (JP125, EFC3APA001, EFC3APA002 and DFC3APA003).

e) A second sucrose gradient ultracentrifugation was performed in order to further purify the virus, while simultaneously splitting it. The centrifugation was performed in the same conditions as the ones described in step d). In particular, the exact same rotors were used, i.e. 400 ml rotors for the experiments JP104, JP115 and JP125 and 1.6 L rotors for the experiments EFC3APA001, EFC3APA002 and DFC3APA003. Sucrose gradient solutions prepared in PBS pH 7.4 (JP125, EFC3APA001, EFC3APA002 and DFC3APA003) or in PO₄ 66 mM pH 7.4 (JP104 and JP115) were loaded to the rotor and then accelerated to the rotational speed of 35000 rpm, so as to form a linear sucrose gradient (5-55% v/w). 2% Triton X-100 (JP125, EFC3APA001, EFC3APA002 and DFC3APA003), or a combination of 1% Triton X-100 and 1% Sodium Deoxycholate (JP115 and JP104) was added to the sucrose layers to achieve a detergent micelles barrier. The whole virus entering this detergent barrier was split. Virus fragments containing the viral membrane proteins hemagglutinin (HA) and neuraminidase (NA) migrated to the micelles density. The remaining virions, some of the host cell protein contaminants and DNA migrated to higher sucrose concentration fractions which are not pooled with the viral proteins. The viral proteins present in the fractions ranging from approximately 18 to 41% sucrose are pooled. This range has been determined on the basis of profiles from SDS-PAGE and from Western blot analysis using anti-HA and anti-MDCK antibodies. The fractions pool containing the viral proteins were in PO₄ 66 mM pH 7.4 (JP104 and JP115), or PBS pH 7.4 (JP125, EFC3APA001, EFC3APA002 and DFC3APA003). This pool was then assayed for the total protein content and diluted to around 250 μg protein/ml with PBS containing 0.01% Tween 80, alpha-tocopheryl hydrogen succinate 0.1 mM and 0.3% Triton X-100 pH 7.4 (JP125, EFC3APA001, EFC3APA002 and DFC3APA003), or PO₄ 66 mM pH 7.4 containing 0.01% Tween 80 and 0.1 mM alpha-tocopheryl hydrogen succinate (JP104 and JP115).

The amount of HA present in the virus preparation to be loaded on the first gradient, i.e. in the concentrated and Benzonase™-treated harvest, was measured by SRD assay, as described below. The amount of HA was also measured after each sucrose gradient ultracentrifugation step (steps d) and e)), so as to calculate the specific HA yield obtained after each sucrose gradient ultracentrifugation step. Accordingly, HA yield 1 corresponds to the percentage of HA recovered from the concentrated and Benzonase™-treated harvest after performing the first sucrose gradient ultracentrifugation, whereas HA yield 2 corresponds to the percentage of HA recovered from the first sucrose gradient ultracentrifugation HA pool after performing the second sucrose gradient ultracentrifugation. The global HA yield row represents the HA recovery measured at the end of each purification process, which takes, thus, into account the loss caused by both sucrose gradient ultracentrifugation steps. Results are presented in Table 1. As indicated in the last row of Table 1, the HA/total protein ratio has also been calculated, which represents the percentage of HA over the total proteins obtained at the end of each purification process. The concentration of total proteins has been measured by the classical Lowry method.

TABLE 1

| | Experiments No. | | | | | |
|---|---|---|---|---|---|---|
| | JP104 | JP115 | JP125 | EFC3APA001 | EFC3APA002 | DFC3APA003 |
| Volume scale (L) | 40 | 20 | 60 | 60 | 60 | 60 |
| (Harvest) | | | | | | |
| Concentrated | 1999 | 2183 | 1300 | 2023 | 1780 | 2129 |
| Harvest (mL) | | | | | | |
| HA amount (mg) | 787 | 749 | 529 | 874 | 751 | 571 |
| in Concentrated | | | | | | |
| Harvest | | | | | | |
| Rotor volume (ml) | 400 | 400 | 400 | 1600 | 1600 | 1600 |
| Ratio HA/rotor | 2.0 | 1.9 | 1.3 | 0.5 | 0.5 | 0.36 |
| volume (mg/ml) | | | | | | |
| HA yield 1 (%) | 50 | 31 | 42 | 95 | 62* | 85 |
| after first sucrose | | | | | | |
| gradient | | | | | | |
| HA yield 2 (%) | 63 | 76 | 83 | 75 | | 87 |
| after second | | | | | | |
| sucrose gradient | | | | | | |
| Global HA yield | 12 | 7 | 10 | 33 | 31 | 37 |
| (%) | | | | | | |
| HA/total proteins | 42 | 34 | 35 | 49 | 42 | 35 |
| (%) | | | | | | |

*HA yield 1 and HA yield 2 values were not available for the experiment EFC3APA002. Instead, shown is the percentage of HA recovered from the concentrated harvest after performing both sucrose gradient ultracentrifugations.

Results-Conclusions

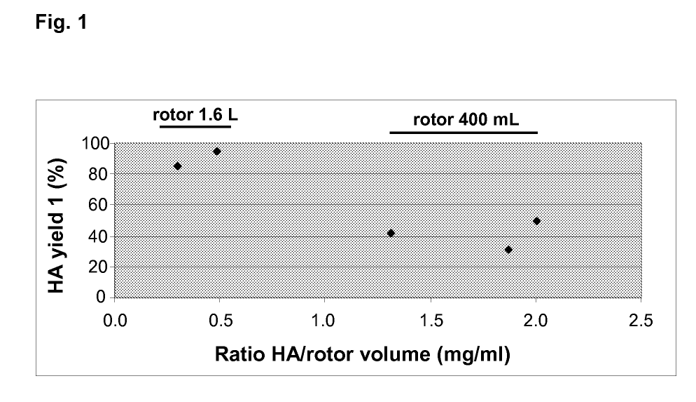

As shown in FIG. 1, where the values of the row Ratio HA/rotor volume (mg/ml) of Table 1 have been plotted against the values of the row HA yield 1(%) after first sucrose gradient of said Table, increasing the volume of sucrose gradient has a positive impact on the first sucrose gradient step by increasing the HA yield of this step (see also row 7 of Table 1 and compare 400 ml rotor columns with 1.6 L rotor columns) by an at least 1.7-fold factor and up to a 3-fold factor. For instance, when comparing experiments JP125 and DFC3APA003, using, respectively, 400 ml rotor and 1.6 L rotor, and in which the amounts of HA loaded on the first sucrose gradient are very similar, 529 mg and 571 mg, respectively, the increase factor is 2. Also, when examining the next-to-last row of Table 1, it can be observed that the global HA yield is improved when using 1.6 L rotors during the first sucrose gradient ultracentrifugation, as compared to using 400 ml rotors.

In conclusion, increasing the first sucrose gradient volume 4 times helps improve the HA yield of this step by a factor ranging from 1.7 to 3 and improve the global HA yield of the influenza virus purification process by a factor of at least 3.

SRD Method Used to Measure HA Content

Glass plates (12.4-10 cm) are coated with an agarose gel containing a concentration of anti-influenza HA serum that is recommended by NIBSC. After the gel has set, 72 sample wells (3 mm diameter) are punched into the agarose. 10 μl of appropriate dilutions of the reference and the sample are loaded in the wells. The plates are incubated for 24 hours at room temperature (20 to 25° C.) in a moist chamber. After that, the plates are soaked overnight with NaCl solution and washed briefly in distilled water. The gel is then pressed and dried. When completely dry, the plates are stained on Coomassie Brillant Blue solution for 10 minutes and destained twice in a mixture of methanol and acetic acid until clearly defined stained zones become visible. After drying the plates, the diameter of the stained zones surrounding antigen wells is measured in two directions at right angles. Alternatively equipment to measure the surface can be used. Dose-response curves of antigen dilutions against the surface are constructed and the results are calculated according to standard slope-ratio assay methods (Finney, D. J. (1952). Statistical Methods in Biological Assay. London: Griffin, Quoted in: Wood, J M, et al (1977). J. Biol. Standard. 5, 237-247)

1. A method for purifying a virus, or a viral antigen thereof, comprising:
a) obtaining a fluid comprising the virus, or a viral antigen thereof, and
b) purifying the fluid by at least one density gradient ultracentrifugation step, wherein the ratio of the amount of virus, or viral antigen thereof, present in the fluid over the density gradient volume is less than 1 mg/mL.

2. (canceled)

3. The method of claim 1, wherein the density gradient is a sucrose gradient.

4. The method of claim 1, wherein the fluid comprising the virus, or a viral antigen thereof, is cell culture medium collected after infection of cells with the virus.

5. The method of claim 1, wherein the fluid comprising the virus, or a viral antigen thereof, is partially purified before undergoing the at least one density gradient ultracentrifugation step.

6. The method of claim 5, wherein the fluid comprising the virus, or a viral antigen thereof, has been clarified.

7. The method of claim 6, wherein clarification is performed by filtration, centrifugation or both.

8. The method of claim 1, wherein the fluid comprising the virus, or a viral antigen thereof, has been concentrated prior to loading it on the density gradient.

9. The method of claim 1, wherein the at least one density gradient ultracentrifugation is performed in a continuous mode.

10. The method of claim 1, further comprising an additional ultracentrifugation step.

11. The method of claim 10, wherein the additional ultracentrifugation step occurs after the at least one density gradient ultracentrifugation step.

12. The method of claim 10, wherein the additional ultracentrifugation step is a density gradient ultracentrifugation step.

13. The method of claim 1, further comprising a splitting step.

14. The method of claim 1, wherein a splitting agent is added to the gradient during the at least one density gradient ultracentrifugation step.

15. The method of claim 12, wherein a splitting agent is added to the gradient during the additional density gradient ultracentrifugation step.

16. The method of claim 13, wherein the splitting step occurs in a batch mode.

17. The method of claim 16, wherein the splitting step occurs after the at least one density gradient ultracentrifugation step.

18. The method of claim 13, wherein the splitting agent is octoxynol-10 (TRITON™ X-100).

19. The method of claim 1, further comprising at least one virus inactivation step.

20. (canceled)

21. The method of claim 4, wherein the cells are mammalian cells.

22. The method of claim 21, wherein the cells are Madin-Darby canine kidney (MDCK) cells.

23. The method of claim 4, wherein the cells are duck embryonic stem cells.

24. The method of claim 1, wherein the virus is influenza virus.

25. A method for the preparation of a vaccine comprising admixing the virus obtained according to claim 1 with a pharmaceutically acceptable carrier.
